(19) United States
(12) Patent Application Publication
(10) Pub. No.: US 2008/0254077 A1
(43) Pub. Date: Oct. 16, 2008

(54) Core/shell particles based on silicone compounds

(75) Inventor: Fanny Prigent, Ceyrat (FR)

Correspondence Address: OLIFF & BERRIDGE, PLC, P.O. BOX 320850, ALEXANDRIA, VA, 22320-4850, US

(73) Assignee: L'OREAL, PARIS (FR)

(21) Appl. No.: 12/003,025

(22) Filed: Dec. 19, 2007

Related U.S. Application Data
(60) Provisional application No. 60/883,172, filed on Jan. 3, 2007.

(30) Foreign Application Priority Data
Dec. 20, 2006 (FR) 06 55678

(51) Int. Cl. A61K 8/11; A61K 9/14; A61K 31/355; A61Q 5/00

(52) U.S. Cl. 424/401; 424/490; 514/458; 424/497

(57) ABSTRACT

The present invention relates to particles of core/shell type suitable for the encapsulation of cosmetic and/or therapeutic lipophilic active agents. It also relates to a process for preparing said particles as well as cosmetic or therapeutic compositions comprising them.

This non provisional application claims the benefit of French Application No. 06 55678 filed on Dec. 20, 2006 and U.S. Provisional Application No. 60/883,172 filed on Jan. 3, 2007.

The present invention relates to particles of core/shell type suitable for the encapsulation of cosmetic and/or therapeutic lipophilic active agents. It also relates to a process for preparing said particles as well as cosmetic or therapeutic compositions comprising them.

It has been common practice for a long time to use, in compositions intended for cosmetic or therapeutic care, active agents in encapsulated form. This type of formulation is generally preferred when it is desired to prevent any potential degradation of the active agent under consideration, which is liable to arise, for example, following contact of this active agent in an unencapsulated form with another component of the composition containing it or, more generally, following prolonged exposure of the composition containing it to the ambient atmosphere. Encapsulation of these active agents thus makes it possible to efficiently reinforce their activity. Examples of hollow particles of microcapsule and nanocapsule type are especially described in documents EP-A-557 489, EP-A-447 318, WO-A-93/25195 and WO-A-93/05753.

These particles generally have a core/shell architecture. The core is usually of oily nature and contains the lipophilic active agent, that is to say an oil or a lipophilic compound other than an oil. As regards the shell, it is generally of organic nature and more particularly polymeric.

However, it is also known that some of these particles may be subjected to uncontrolled release phenomenon, for example untimely release or release that may be termed spurious insofar as it is low-level but continuous release. In point of fact, this type of release is generally caused by partial fluidization of the polymer forming the shell of the particles. This fluidization may especially be the consequence of exposure of the particles to a rise in temperature.

For obvious reasons, this stability defect, manifested by certain polymers, is particularly detrimental as regards the efficacy.

The object of the present invention is, precisely, to propose novel particles that are suitable for encapsulating active agents and that are in particular free of the abovementioned drawback.

Consequently, according to one of its aspects, the present invention relates to particles of core/shell type with a lipophilic core, characterized in that their shell is at least partly formed from a silicone polymer film that is the product of in situ reaction between at least one compound X and at least one compound Y with at least one of the compounds X and Y being a polyorganosiloxane and said reaction being of hydrosilylation type performed in the presence of a catalyst, or of condensation or crosslinking type performed in the presence of a peroxide.

For the purposes of the present invention, the expression “in situ reaction” means that the reaction between compounds X and Y takes place at the time of formation of the particles.

Advantageously, the core of the particles comprises at least one oil and/or one lipophilic cosmetic or therapeutic active agent.

According to one embodiment, said particles may be particles whose shell is also at least partly coated with a lamellar phase.

According to another of its aspects, the present invention relates to an aqueous dispersion of said particles.

According to yet another of its aspects, the present invention relates to cosmetic and/or therapeutic compositions, especially intended for caring for and/or making up keratin material(s), containing, in a physiologically acceptable medium, at least one particle in accordance with the invention.

According to yet another of its aspects, the present invention relates to the cosmetic use of said particles as vehicle for lipophilic active agent(s) in a composition, especially a cosmetic composition and more particularly for caring for the skin, and for photoprotecting and also making up keratin material(s).

According to yet another of its aspects, the present invention relates to the use of said particles as vehicle for lipophilic active agent(s) for the preparation of compositions, especially therapeutic compositions.

The invention is also directed towards a cosmetic method for treating keratin material(s), comprising at least the application to a keratin material of particles or of a composition in accordance with the invention.

According to yet another of its aspects, the present invention relates to a process for preparing particles of core/shell type whose core is lipophilic and whose shell is formed at least partly from a silicone polymer film, said process comprising at least the steps consisting in

1) dissolving, in at least one volatile organic solvent, at least one lipophilic compound, a compound X and a compound Y, with at least one of the compounds X and Y being a polyorganosiloxane and compounds X and Y being under conditions not suitable for their interaction, and

2) placing the organic phase from step 1 in contact with an aqueous phase containing, where appropriate, at least one surfactant, under conditions suitable for the formation of lipophilic droplets and for the reaction of compounds X and Y according to a reaction of hydrosilylation type in the presence of a catalyst, or of condensation or crosslinking type in the presence of a peroxide, to form a silicone polymer film at the surface of said droplets.

The particles thus formed may be isolated by evaporation of the organic solvent(s).

According to one embodiment, step 2 is performed in the presence of a catalyst if it is necessary for the interaction of compounds X and Y.

According to another particular embodiment, the organic phase of step 1 may also comprise a compound capable of forming a coat of lamellar phase type at the surface of the expected particles and/or an additional lipophilic active agent, that is to say an active agent different from the lipophilic compound.

According to a first embodiment variant, the organic solvent is sparingly water-miscible, for instance dichloromethane, and step 2 corresponds to an emulsification of the organic phase of step 1 in the aqueous phase in the form of droplets, at the surface of which compounds X and Y interact to form the polymer film constituting the shell of the corresponding particles.

This particle preparation technique is especially described in patent FR 2 864 900.

According to a second embodiment variant, the organic solvent is water-miscible and step 2 corresponds to solvent tipping, during which the diffusion of the aqueous phase into the organic phase causes the formation of droplets, at the surface of which compounds X and Y interact to produce the polymer film forming the shell of the corresponding particles.

This particle preparation technique is especially described in document EP 0 274 961.

Unexpectedly, and as demonstrated by the examples below, the inventors have thus found that a polymer film produced by in situ reaction of a compound X with at least one compound Y as defined below, that is to say whose formation takes place at the same time as that of the particles of core/shell type of which it constitutes the shell, leads to particles that show increased stability over time.

Compounds X and Y, more specifically described below, are compounds that are known for generally interacting at room temperature and atmospheric pressure and thus for forming silicone polymer films. These compounds are partly described in documents WO 01/96450 and GB 2 407 496. However, to the inventors' knowledge, these compounds have hitherto never been used for obtaining particles in accordance with the invention.

### DEFINITION OF THE PARTICLES

The particles according to the invention may have a mean particle diameter (or “size”) ranging from 50 nm to 100 μm.

This diameter may especially be controlled according to a measuring technique based on dynamic light scattering, for example using a BI90+® machine from Brookhaven Instrument or a Malvern Mastersizer 2000 machine (suitable for diameters of greater than 2 μm).

According to a first embodiment variant, the particles according to the invention may be microcapsules and may thus have a mean particle diameter ranging from 1 μm to 100 μm.

According to a second variant, they are nanocapsules and they then have a mean particle diameter ranging from 50 nm to 1 μm.

As stated previously, the particles according to the invention have an architecture of core/shell type and have a core of lipophilic nature.

### SHELL OF THE PARTICLES ACCORDING TO THE INVENTION

The shell of the particles according to the invention is at least partly formed from, and advantageously constituted of, a silicone polymer film formed in situ by reacting at least one compound X and at least one compound Y, where appropriate in the presence of a catalyst or a peroxide, at least one of these two compounds being a polyorganosiloxane.

Compounds X and Y

Silicone compound means a polyorganosiloxane compound, i.e. comprising at least two organosiloxane units, for example at least 5 organosiloxane units, notably at least 10 organosiloxane units. According to a particular embodiment, at least one of compounds X and Y, or compounds X and compounds Y are polyorganosiloxanes. Compounds X and Y can be aminated or non-aminated.

According to another embodiment, at least one of compounds X and Y is a polymer whose main chain is formed primarily of organosiloxane units.

The inventors have in fact noted that the compounds comprising a single organosiloxane unit form, by polymerization, a very rigid network, leading to particles having very hard shells.

On the other hand, by using at least one compound X or Y of polyorganosiloxane type, the shell of the resulting particles is more supple, and the compositions comprising these particles have better cosmetic properties, notably better softness when applying to the skin.

Among the silicone compounds mentioned below, some may display both film-forming and adhesive properties, depending for example on their proportion of silicone or depending on whether they are used mixed with a particular additive. It is therefore possible to adjust the film-forming properties or the adhesive properties of said compounds according to the proposed use, which is the case in particular for the so-called “room temperature vulcanization” reactive elastomeric silicones.

Compounds X and Y can react with each other at a temperature varying between room temperature and 180° C. Advantageously, compounds X and Y are capable of reacting together at room temperature (20±5° C.) and atmospheric pressure, by a hydrosilylation reaction in the presence of a catalyst, or a condensation reaction, if appropriate in the presence of a catalyst, or a crosslinking reaction in the presence of a peroxide.

Polar Groups

According to a particular embodiment, at least one of compounds X and Y, for example compound X, bears at least one polar group that is capable of forming at least one hydrogen bond with keratin materials.

By polar group, we mean a group having carbon atoms and hydrogen atoms in its chemical structure and at least one heteroatom (such as O, N, S and P), such that said group is able to establish at least one hydrogen bond with keratin materials.

Compounds bearing at least one group that can form a hydrogen bond are particularly advantageous, as they endow the compositions containing them with better adherence on keratin materials.

The polar group or groups borne by at least one of compounds X and Y is/are able to establish a hydrogen bond, and include either a hydrogen atom bound to an electronegative atom, or an electronegative atom for example an oxygen, nitrogen or sulfur atom. When the group has a hydrogen atom bound to an electronegative atom, the hydrogen atom can interact with another electronegative atom borne for example by another molecule, such as keratin, to form a hydrogen bond. When the group has an electronegative atom, the electronegative atom can interact with a hydrogen atom bound to an electronegative atom borne for example by another molecule, such as keratin, to form a hydrogen bond.

Advantageously, these polar groups can be selected from the following groups:
carboxylic acids —COOH,
alcohols, such as: —CH₂OH or —CH(R)OH, R being an alkyl radical having from 1 to 6 carbon atoms,
amino of formula —NR₁R₂, in which R₁ and R₂, which may be identical or different, represent an alkyl radical having from 1 to 6 carbon atoms or one of R₁ or R₂ denotes a hydrogen atom, and the other one of R₁ and R₂ represents an alkyl radical having from 1 to 6 carbon atoms,
pyridino,
amido of formula —NH—COR′ or —CO—NH—R′ in which R′ represents a hydrogen atom or an alkyl radical having from 1 to 6 carbon atoms,
pyrrolidino preferably selected from the groups of formula:

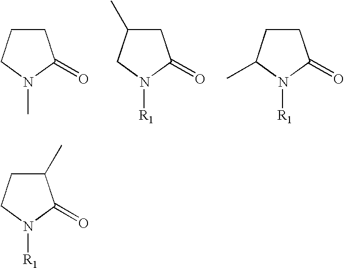

R₁ being an alkyl radical having from 1 to 6 carbon atoms,
carbamoyl of formula —O—CO—NH—R′ or —NH—CO—OR′, R′ being as defined above,
thiocarbamoyl such as —O—CS—NH—R′ or —NH—CS—OR′, R′ being as defined above,
ureyl such as —NR′—CO—N(R′)₂, the groups R′, which may be identical or different, being as defined above,
sulfonamido such as —NR′—S(═O)₂—R′, R′ corresponding to the above definition.

Preferably, these polar groups are present at a content less than or equal to 10% by weight relative to the weight of each compound X or Y, preferably less than or equal to 5% by weight, for example at a content ranging from 1% to 3% by weight.

The polar group or groups can be located in the main chain of compound X and/or Y or can be pendent from the main chain or located at the ends of the main chain of compound X and/or Y.

1—Compounds X and Y Capable of Reacting by Hydrosilylation

According to one embodiment, the invention relates to particles of core/shell type having a lipophilic core, characterized in that their shell is formed at least partly by a silicone polymer film which is the product of in situ reaction between at least one compound X and at least one compound Y, at least one of compounds X and Y being a polyorganosiloxane and said reaction being of hydrosilylation type carried out in the presence of a catalyst.

According to this embodiment, compounds X and Y are capable of reacting by hydrosilylation in the presence of a catalyst, said reaction being represented schematically in a simplified manner as follows:

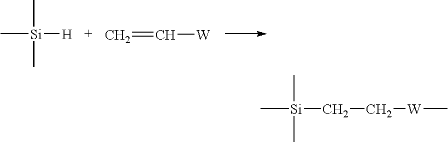

with W representing a carbon chain and/or silicone chain containing one or more unsaturated aliphatic groups.

In this case, compound X can be selected from silicone compounds comprising at least two unsaturated aliphatic groups. As an example, compound X can be a polyorganosiloxane comprising a silicone main chain whose unsaturated aliphatic groups are pendent from the main chain (side group) or located at the ends of the main chain of the compound (end group). These particular compounds will be called, hereinafter, polyorganosiloxanes with unsaturated aliphatic groups.

According to one embodiment, compound X and/or compound Y bear at least one polar group, as described above, capable of forming at least one hydrogen bond with keratin materials. This polar group is advantageously carried by compound X, which has at least two unsaturated aliphatic groups.

According to one embodiment, compound X is selected from the polyorganosiloxanes comprising at least two unsaturated aliphatic groups, for example two or three vinyl or allyl groups, each attached to a silicon atom.

According to an advantageous embodiment, compound X is selected from the polyorganosiloxanes containing siloxane units of formula:

$${R}_{m} {R}^{′} SiO ( \frac{3 - m}{2} ) \qquad ( I )$$

in which:
R represents a linear or cyclic, monovalent hydrocarbon group, having from 1 to 30 carbon atoms, preferably from 1 to 20, and better still from 1 to 10 carbon atoms, for example a short-chain alkyl radical, comprising for example from 1 to 10 carbon atoms, in particular a methyl radical or alternatively a phenyl group, preferably a methyl radical,
m is equal to 1 or 2 and
R′ represents:
   an unsaturated aliphatic hydrocarbon group having from 2 to 10, preferably from 3 to 5 carbon atoms, for example a vinyl group or a group —R″—CH═CHR′″ in which R″ is a divalent aliphatic hydrocarbon chain, having from 1 to 8 carbon atoms, bound to the silicon atom and R′″ is a hydrogen atom or an alkyl radical having from 1 to 4 carbon atoms, preferably a hydrogen atom; we may mention, as group R′, the vinyl and allyl groups and mixtures thereof; or
   an unsaturated cyclic hydrocarbon group having from 5 to 8 carbon atoms, for example a cyclohexenyl group.

Preferably R′ is an unsaturated aliphatic hydrocarbon group, preferably a vinyl group.

According to one embodiment, R represents an alkyl radical having from 1 to 10 carbon atoms or alternatively a phenyl group, and preferably a methyl radical, and R′ is a vinyl group.

According to a particular embodiment, the polyorganosiloxane also contains units of formula:

$${R}_{n} SiO ( \frac{4 - n}{2} ) \qquad ( II )$$

in which R is a group as defined previously, and n is equal to 1, 2 or 3.

According to a variant, compound X can be a silicone resin comprising at least two ethylenic unsaturations, said resin being capable of reacting with compound Y by hydrosilylation in the presence of a catalyst. We may mention for example the resins of type MQ or MT which themselves bear —CH═CH₂ unsaturated reactive end groups.

These resins are crosslinked organosiloxane polymers.

The class of the silicone resins is known by the name “MDTQ”, the resin being described in relation to the different siloxane monomer units that it contains, each of the letters “MDTQ” characterizing a type of unit.

The letter M represents the monofunctional unit of formula (CH₃)₃SiO_{1/2}, the silicon atom being bound to a single oxygen atom in the polymer comprising said unit.

The letter D denotes a bifunctional unit (CH₃)₂SiO_{2/2} in which the silicon atom is bound to two oxygen atoms.

The letter T represents a trifunctional unit of formula (CH₃)SiO_{3/2}.

In units M, D, T defined above, at least one of the methyl groups can be substituted with a group R other than the methyl group, such as a hydrocarbon radical (notably alkyl) having from 2 to 10 carbon atoms or a phenyl group or alternatively a hydroxyl group.

Finally, the letter Q denotes a tetrafunctional unit SiO_{4/2} in which the silicon atom is bound to four hydrogen atoms which are themselves attached to the rest of the polymer. As examples of said resins, we may mention the MT silicone resins such as poly(phenyl-vinylsilsesquioxane) such as that marketed under the reference SST-3PV1 by the company Gelest.

Preferably, compounds X have from 0.01% to 1% by weight, of unsaturated aliphatic groups.

Advantageously, compound X is selected from the polyorganopolysiloxanes, notably those comprising the siloxane units (I) and optionally (II) described previously.

Compound Y preferably has at least two free Si—H groups (hydrogenosilane groups).

Compound Y can be selected advantageously from the polyorganosiloxanes comprising at least one alkylhydrogenosiloxane unit of the following formula:

$${R}_{p} HSiO ( \frac{3 - p}{2} ) \qquad ( III )$$

in which:

R represents a linear or cyclic, monovalent hydrocarbon group, having from 1 to 30 carbon atoms, for example an alkyl radical having from 1 to 30 carbon atoms, preferably from 1 to 20 and better still from 1 to 10 carbon atoms, in particular a methyl radical, or alternatively a phenyl group and p is equal to 1 or 2. Preferably R is a hydrocarbon group, preferably methyl.

These polyorganosiloxane compounds Y with alkylhydrogenosiloxane units can additionally contain units of formula:

$${R}_{n} SiO ( \frac{4 - n}{2} ) \qquad ( II )$$

as defined above.

Compound Y can be a silicone resin comprising at least one unit selected from the units M, D, T, Q as defined above and comprising at least one Si—H group such as the poly(methyl-hydridosilsesquioxane) marketed under the reference SST-3MH1.1 by the company Gelest.

Preferably, these polyorganosiloxane compounds Y have from 0.5% to 2.5% by weight of Si—H groups.

Advantageously, the radicals R represent a methyl group in formulae (I), (II), (III) above.

Preferably, these polyorganosiloxanes Y have end groups of formula (CH₃)₃SiO_{1/2}.

Advantageously, the polyorganosiloxanes Y have at least two alkylhydrogenosiloxane units of formula —(H₃C)(H)SiO— and optionally include —(H₃C)₂SiO— units.

These polyorganosiloxane compounds Y with hydrogenosilane groups are described for example in document EP 0 465 744.

According to one variant, compound X is selected from the organic oligomers or polymers (by organic, we mean compounds whose main chain is not a silicone chain, preferably compounds not containing silicon atoms) or from hybrid organic/silicone polymers or oligomers, said oligomers or polymers bearing at least 2 unsaturated reactive aliphatic groups, compound Y being selected from the polyorganosiloxanes Y with hydrogenosilane groups mentioned above.

According to one embodiment, the organic or hybrid organic/silicone compounds X bearing at least 2 unsaturated reactive aliphatic groups, have at least one polar group as described above.

Compound X, of organic nature, can then be selected from the vinylic, (meth)acrylic polymers or oligomers, polyesters, polyurethanes and/or polyureas, polyethers, perfluoropolyethers, polyolefins such as polybutene, polyisobutylene, dendrimers or organic hyperbranched polymers, or mixtures thereof.

In particular, the organic polymer or the organic moiety of the hybrid polymer can be selected from the following polymers:

a) polyesters with ethylenic unsaturation(s):

This is a group of polymers of the polyester type having at least 2 ethylenic double bonds, randomly distributed in the main chain of the polymer. These unsaturated polyesters are obtained by polycondensation of a mixture:
of linear or branched aliphatic or cycloaliphatic dicarboxylic acids notably having 3 to 50 carbon atoms, preferably from 3 to 20 and better still from 3 to 10 carbon atoms, such as adipic acid or sebacic acid, of aromatic dicarboxylic acids notably having from 8 to 50 carbon atoms, preferably from 8 to 20 and better still from 8 to 14 carbon atoms, such as phthalic acids, notably terephthalic acid, and/or of dicarboxylic acids derived from dimers of fatty acids with ethylenic unsaturations such as the dimers of oleic or linoleic acids described in application EP-A-959 066 (paragraph [0021]) marketed under the designations Pripol® by the company Uniqema or Empol® by the company Henkel, all said diacids having to be free from polymerizable ethylenic double bonds,
of linear or branched aliphatic or cycloaliphatic diols notably having from 2 to 50 carbon atoms, preferably from 2 to 20 and better still from 2 to 10 carbon atoms, such as ethylene glycol, diethylene glycol, propylene glycol, 1,4-butanediol or cyclohexanedimethanol, of aromatic diols having from 6 to 50 carbon atoms, preferably from 6 to 20 and better still from 6 to 15 carbon atoms such as bisphenol A and bisphenol B, and/or of diol dimers resulting from reduction of dimers of fatty acids as defined previously, and
of one or more dicarboxylic acids or their anhydrides having at least one polymerizable ethylenic double bond and having from 3 to 50 carbon atoms, preferably from 3 to 20 and better still from 3 to 10 carbon atoms, such as maleic acid, fumaric acid or itaconic acid.

b) polyesters with (meth)acrylate side and/or end groups:

This is a group of polymers of the polyester type obtained by polycondensation of a mixture:
of linear or branched aliphatic or cycloaliphatic dicarboxylic acids notably having from 3 to 50 carbon atoms, preferably from 3 to 20 and better still from 3 to 10 carbon atoms, such as adipic acid or sebacic acid, of aromatic dicarboxylic acids notably having from 8 to 50 carbon atoms, preferably from 8 to 20 and better still from 8 to 14 carbon atoms, such as phthalic acids, notably terephthalic acid, and/or of dicarboxylic acids derived from dimers of fatty acids with an ethylenic unsaturation such as the dimers of oleic or linoleic acids described in application EP-A-959 066 (paragraph [0021]) marketed under the designations Pripol® by the company Uniqema or Empol® by the company Henkel, all said diacids having to be free from polymerizable ethylenic double bonds,
of linear or branched aliphatic or cycloaliphatic diols notably having from 2 to 50 carbon atoms, preferably from 2 to 20 and better still from 2 to 10 carbon atoms, such as ethylene glycol, diethylene glycol, propylene glycol, 1,4-butanediol or cyclohexanedimethanol, of aromatic diols having from 6 to 50 carbon atoms, preferably from 6 to 20 and better still from 6 to 15 carbon atoms such as bisphenol A and bisphenol B, and
of at least one ester of (meth)acrylic acid and of a diol or polyol having from 2 to 20 carbon atoms, preferably from 2 to 6 carbon atoms, such as 2-hydroxyethyl (meth)acrylate, 2-hydroxypropyl(meth)acrylate and glycerol methacrylate.

These polyesters differ from those described above in section a) by the fact that the ethylenic double bonds are not located in the main chain but on side groups or at the end of the chains. These ethylenic double bonds are those of the (meth)acrylate groups present in the polymer.

Such polyesters are marketed for example by the company UCB under the designations Ebecryl® (Ebecryl® 450: molecular weight 1600, on average 6 acrylate functions per molecule, Ebecryl® 652: molecular weight 1500, on average 6 acrylate functions per molecule, Ebecryl® 800: molecular weight 780, on average 4 acrylate functions per molecule, Ebecryl® 810: molecular weight 1000, on average 4 acrylate functions per molecule, Ebecryl® 50 000: molecular weight 1500, on average 6 acrylate functions per molecule).

c) polyurethanes and/or polyureas with (meth)acrylate groups, obtained by polycondensation:
of aliphatic, cycloaliphatic and/or aromatic diisocyanates, triisocyanates and/or polyisocyanates notably having from 4 to 50, preferably from 4 to 30 carbon atoms, such as hexamethylenediisocyanate, isophoronediisocyanate, toluenediisocyanate, diphenylmethanediisocyanate or isocyanurates of formula:

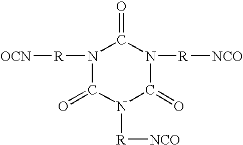

resulting from the trimerization of 3 molecules of diisocyanates OCN—R—CNO, where R is a linear, branched or cyclic hydrocarbon radical having from 2 to 30 carbon atoms;
of polyols, notably of diols, free from polymerizable ethylenic unsaturations, such as 1,4-butanediol, ethylene glycol or trimethylolpropane, and/or of polyamines, notably of aliphatic, cycloaliphatic and/or aromatic diamines, notably having from 3 to 50 carbon atoms, such as ethylenediamine or hexamethylenediamine, and
of at least one ester of (meth)acrylic acid and of a diol or polyol having from 2 to 20 carbon atoms, preferably from 2 to 6 carbon atoms, such as 2-hydroxyethyl (meth)acrylate, 2-hydroxypropyl(meth)acrylate and glycerol methacrylate.

These polyurethanes/polyureas with acrylate groups are marketed for example under the designation SR 368 (tris(2-hydroxyethyl)isocyanurate-triacrylate) or Craynor® 435 by the company Cray Valley, or under the designation Ebecryl® by the company UCB (Ebecryl® 210: molecular weight 1500, 2 acrylate functions per molecule, Ebecryl® 230: molecular weight 5000, 2 acrylate functions per molecule, Ebecryl® 270: molecular weight 1500, 2 acrylate functions per molecule, Ebecryl® 8402: molecular weight 1000, 2 acrylate functions per molecule, Ebecryl® 8804: molecular weight 1300, 2 acrylate functions per molecule, Ebecryl® 220: molecular weight 1000, 6 acrylate functions per molecule, Ebecryl® 2220: molecular weight 1200, 6 acrylate functions per molecule, Ebecryl® 1290: molecular weight 1000, 6 acrylate functions per molecule, Ebecryl® 800: molecular weight 800, 6 acrylate functions per molecule).

We may also mention the water-soluble aliphatic diacrylate polyurethanes marketed under the designations Ebecryl® 2000, Ebecryl® 2001 and Ebecryl® 2002, and the diacrylate polyurethanes in aqueous dispersion marketed under the trade names IRR® 390, IRR® 400, IRR® 422 IRR® 424 by the company UCB.

d) polyethers with (meth)acrylate groups obtained by esterification, by (meth)acrylic acid, of the hydroxyl end groups of homopolymers or of C₁₋₄ alkylene glycol copolymers, such as polyethylene glycol, polypropylene glycol, copolymers of ethylene oxide and of propylene oxide preferably having a weight-average molecular weight below 10 000, polyethoxylated or polypropoxylated trimethylolpropane.

Di(meth)acrylate polyoxyethylenes of suitable molecular weight are marketed for example under the designations SR 259, SR 344, SR 610, SR 210, SR 603 and SR 252 by the company Cray Valley or under the designation Ebecryl® 11 by UCB. Polyethoxylated trimethylolpropane triacrylates are marketed for example under the designations SR 454, SR 498, SR 502, SR 9035, SR 415 by the company Cray Valley or under the designation Ebecryl® 160 by the company UCB. Polypropoxylated trimethylolpropane triacrylates are marketed for example under the designations SR 492 and SR 501 by the company Cray Valley.

e) epoxyacrylates obtained by reaction between
at least one diepoxide selected for example from:
   (i) bisphenol A diglycidyl ether,
   (ii) a diepoxy resin resulting from the reaction between bisphenol A diglycidyl ether and epichlorohydrin,
   (iii) an epoxyester resin with [symbol] diepoxy end groups resulting from the condensation of a dicarboxylic acid having from 3 to 50 carbon atoms with a stoichiometric excess of (i) and/or (ii),
   (iv) an epoxyether resin with [symbol] -diepoxy end groups resulting from the condensation of a diol having from 3 to 50 carbon atoms with a stoichiometric excess of (i) and/or (ii),
   (v) natural or synthetic oils bearing at least 2 epoxide groups, such as epoxidized soya oil, epoxidized linseed oil and epoxidized vemonia oil,
   (vi) a phenol-formaldehyde polycondensate (Novolac® resin), of which the end groups and/or side groups have been epoxidized, and
one or more carboxylic acids or carboxylic polyacids having at least one ethylenic double bond at α,β of the carboxyl group such as (meth)acrylic acid or crotonic acid or esters of (meth)acrylic acid and of a diol or polyol having from 2 to 20 carbon atoms, preferably from 2 to 6 carbon atoms such as 2-hydroxyethyl(meth)acrylate.

Such polymers are marketed for example under the designations SR 349, SR 601, CD 541, SR 602, SR 9036, SR 348, CD 540, SR 480, CD 9038 by the company Cray Valley, under the designations Ebecryl® 600 and Ebecryl® 609, Ebecryl® 150, Ebecryl® 860, Ebecryl® 3702 by the company UCB and under the designations Photomer® 3005 and Photomer® 3082 by the company Henkel.

f) (C₁₋₅₀ alkyl) poly(meth)acrylates, said alkyl being linear, branched or cyclic, bearing at least two functions with ethylenic double bond carried by the lateral and/or terminal hydrocarbon chains.

Such copolymers are marketed for example under the designations IRR® 375, OTA® 480 and Ebecryl® 2047 by the company UCB.

g) polyolefins such as polybutene, polyisobutylene,

h) perfluoropolyethers with acrylate groups obtained by esterification, for example by (meth)acrylic acid, of perfluoropolyethers bearing hydroxyl side and/or end groups.

These α,ω-diol perfluoropolyethers are described notably in EP-A-1 057 849 and are marketed by the company Ausimont under the designation Fomblin® Z Diol.

i) dendrimers and hyperbranched polymers bearing (meth)acrylate or (meth)acrylamide end groups obtained respectively by esterification or amidation of dendrimers and of hyperbranched polymers with hydroxyl or amino terminal functions, by (meth)acrylic acid.

The dendrimers (from the Greek dendron=tree) are “tree-like” polymer molecules, i.e. highly branched, invented by D. A. Tomalia and his team at the beginning of the 1990s (Donald A. Tomalia et al., Angewandte Chemie, Int. Engl. Ed., Vol. 29, No. 2, pages 138-175). They are structures constructed around a, generally polyvalent, central unit. Branched chain-extending units are arranged according to a perfectly defined structure around this central unit, thus giving rise to symmetrical, monodispersed macromolecules having a well-defined chemical and stereochemical structure. Dendrimers of the polyamidoamine type are marketed for example under the name Starburst® by the company Dendritech.

The hyperbranched polymers are polycondensates, generally of the polyester, polyamide or polyethyleneamine type, obtained from multifunctional monomers, which have a tree-like structure similar to that of the dendrimers but far less regular than the latter (see for example WO-A-93/17060 and WO 96/12754).

The company Perstorp markets hyperbranched polyesters under the name Boltom®. Hyperbranched polyethyleneamines are available under the name Comburst® from the company Dendritech. Hyperbranched poly(esteramide)s with hydroxyl end groups are marketed by the company DSM under the name Hybrane®.

These dendrimers and hyperbranched polymers, esterified or amidated by acrylic and/or methacrylic acid, differ from the polymers described in sections a) to h) above by the very large number of ethylenic double bonds present. This increased functionality, generally greater than 5, makes them particularly useful in enabling them to act as a “crosslinking node”, i.e. a multiple crosslinking site.

It is therefore possible to use these dendritic and hyperbranched polymers in association with one or more of the above polymers and/or oligomers a) to h).

1a—Additional Reactive Compounds

According to one embodiment, the compositions containing compound X and/or Y can additionally comprise an additional reactive compound such as:
organic or mineral particles having on their surface at least 2 unsaturated aliphatic groups—we may mention for example the silicas surface-treated for example with silicone compounds with vinylic groups such as for example cyclotetramethyltetravinylsiloxane-treated silica,
silazane compounds such as hexamethyldisilazane.

1b—Catalyst

The hydrosilylation reaction takes place in the presence of a catalyst which can be present with one or other of the compounds X or Y or can be present on its own. For example, this catalyst can be present in the composition in an encapsulated form if the two compounds X and Y, which it must cause to interact, are present in this same composition in an unencapsulated form or conversely it can be contained there in an unencapsulated form if at least one of compounds X and Y is present in the composition in an encapsulated form. The catalyst is preferably based on platinum or tin.

We may mention for example platinum-based catalysts deposited on a support of silica gel or of powdered charcoal, platinum chloride, salts of platinum and of chloroplatinic acids.

The chloroplatinic acids are preferably used in hexahydrate or anhydrous form, which are easily dispersible in organosilicone media.

We may also mention platinum complexes, such as those based on chloroplatinic acid hexahydrate and divinyl tetramethyldisiloxane.

The catalyst can be present at a content in the range from 0.0001% to 20% by weight relative to the total weight of the composition containing it.

Compounds X and/or Y can be combined with polymerization inhibitors or retarders, and more particularly inhibitors of the catalyst. Non-limitatively, we may mention cyclic polymethylvinylsiloxanes, and in particular tetravinyl tetramethyl cyclotetrasiloxane, acetylenic alcohols, preferably volatile, such as methylisobutynol.

The presence of ionic salts, such as sodium acetate, can have an influence on the rate of polymerization of the compounds.

As an example of a combination of compounds X and Y reacting by hydrosilylation in the presence of a catalyst, we may mention the following references offered by the company Dow Corning: DC 7-9800 Soft Skin Adhesive Parts A & B, as well as the combination of the following mixtures A and B prepared by Dow Corning:

MIXTURE A:

| Ingredient (INCI name) | CAS No. | Contents (%) | Function |
|---|---|---|---|
| Dimethyl Siloxane, | 68083-19-2 | 55-95 | Polymer |
| Dimethylvinylsiloxy- | | | |
| terminal | | | |
| Silica Silylate | 68909-20-6 | 10-40 | Filler |
| 1,3-Diethenyl-1,1,3,3- | 68478-92-2 | Trace | Catalyst |
| Tetramethyldisiloxane | | | |
| complexes | | | |
| Tetramethyldivinyldisiloxane | 2627-95-4 | 0.1-1 | Polymer |

MIXTURE B:

| Ingredient (INCI name) | CAS No. | Contents (%) | Function |
|---|---|---|---|
| Dimethyl Siloxane, | 68083-19-2 | 55-95 | Polymer |
| Dimethylvinylsiloxy- | | | |
| terminal | | | |
| Silica Silylate | 68909-20-6 | 10-40 | Filler |
| Dimethyl, | 68037-59-2 | 1-10 | Polymer |
| Methylhydrogen | | | |
| Siloxane, trimethylsiloxy- | | | |
| terminal | | | |

Advantageously, compounds X and Y are selected from silicone compounds capable of reacting by hydrosilylation in the presence of a catalyst; in particular compound X is selected from the polyorganosiloxanes comprising units of formula (I) described above and compound Y is selected from organosiloxanes comprising alkylhydrogenosiloxane units of formula (III) described above.

According to a particular embodiment, compound X is a polydimethylsiloxane with vinylic end groups, and compound Y is a polymethylhydrogenosiloxane.

2/Compounds X and Y Capable of Reacting by Condensation

According to one embodiment, the invention relates to particles of core/shell type having a lipophilic core, characterized in that their shell is formed at least partly by a silicone polymer film which is the product of in situ reaction between at least one compound X and at least one compound Y, at least one of compounds X and Y being a polyorganosiloxane and said reaction being of condensation type carried out, if applicable, in the presence of a catalyst.

According to this embodiment, compounds X and Y are capable of reacting by condensation, either in the presence of water (hydrolysis) by reaction of 2 compounds bearing alkoxysilane groups, or by so-called “direct” condensation by reaction of a compound bearing alkoxysilane group(s) and a compound bearing silanol group(s) or by reaction of 2 compounds bearing silanol group(s).

When the condensation is carried out in the presence of water, the latter can in particular be the ambient humidity, the residual water of the skin, of the lips, of the eyelashes and/or of the nails, or water from an external source, for example by prior moistening of the keratin material (for example by an atomizer, by natural or artificial tears).

In this manner of reaction by condensation, compounds X and Y, which may be identical or different, can therefore be selected from silicone compounds whose main chain contains at least two alkoxysilane groups and/or at least two silanol (Si—OH) side groups or end groups.

According to one embodiment, compound X and/or compound Y bears at least one polar group, as described above, capable of forming at least one hydrogen bond with keratin materials.

According to an advantageous embodiment, compounds X and/or Y are selected from the polyorganosiloxanes comprising at least two alkoxysilane groups. By “alkoxysilane group”, we mean a group comprising at least one —Si—OR moiety, R being an alkyl group having from 1 to 6 carbon atoms.

Compounds X and Y are notably selected from the polyorganosiloxanes comprising alkoxysilane end groups, more specifically those which have at least 2 alkoxysilane end groups, preferably trialkoxysilane end groups.

These compounds X and/or Y preferably mostly comprise units of formula:

R⁹ₛSiO_{(4-s)/2}, (IV)

in which the groups R⁹ represent, independently of one another, a radical selected from alkyl groups having from 1 to 6 carbon atoms, the phenyl group, the fluoroalkyl groups, and s is equal to 0, 1, 2 or 3. Preferably, groups R⁹ represent, independently of one another, an alkyl group having from 1 to 6 carbon atoms. As alkyl group, we may notably mention methyl, propyl, butyl, hexyl and mixtures thereof, preferably methyl or ethyl. As fluoroalkyl group, we may mention 3,3,3-trifluoropropyl.

According to a particular embodiment, compounds X and Y, which may be identical or different, are polyorganosiloxanes comprising units of formula:

(R⁹₂SiO₂)_{f}— (V)

in which R⁹ is as described above, preferably R⁹ is a methyl radical, and f is such that the polymer advantageously has a viscosity at 25° C. in the range from 0.5 to 3000 Pa·s, preferably in the range from 5 to 150 Pa·s; for example f can range from 2 to 5000, preferably from 3 to 3000, and more preferably from 5 to 1000.

These polyorganosiloxane compounds X and Y contain at least 2 trialkoxysilane end groups per molecule of polymer, said groups having the following formula

-ZSiR¹ₓ(OR)₃₋ₓ, (VI)

in which:

the radicals R represent, independently, a methyl, ethyl, n-propyl, isopropyl, n-butyl, sec-butyl, isobutyl group, preferably a methyl or ethyl group,

R¹ is a methyl or ethyl group,

x is equal to 0 or 1, preferably x is equal to 0 and

Z is selected from: the divalent hydrocarbon groups without an ethylenic unsaturation and having from 1 to 18 carbon atoms, preferably from 2 to 18 carbon atoms (alkylene groups), the combinations of divalent hydrocarbon radicals and of siloxane segments of the following formula (IX):

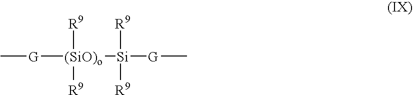

R⁹ being as described above, G is a divalent hydrocarbon radical without an ethylenic unsaturation and having from 1 to 18 carbon atoms, preferably from 2 to 18 carbon atoms and c is an integer in the range from 1 to 6.

Z and G can notably be selected from the alkylene groups such as methylene, ethylene, propylene, butylene, pentylene, hexylene, the arylene groups such as phenylene.

Preferably, Z is an alkylene group, and more preferably ethylene.

These polymers can have on average at least 1.2 trialkoxysilane end groups or terminal chains per molecule, and preferably on average at least 1.5 trialkoxysilane end groups per molecule. These polymers that can have at least 1.2 trialkoxysilane end groups per molecule, some can include other types of end groups such as end groups of formula CH₂═CH—SiR⁹₂₋ or of formula R⁶₃—Si—, in which R⁹ is as defined previously and each group R⁶ is selected independently from the R⁹ or vinyl groups. As examples of said end groups, we may mention the trimethoxysilane, triethoxysilane, vinyldimethoxysilane and vinylmethyloxyphenylsilane groups.

Such polymers are notably described in documents U.S. Pat. No. 3,175,993, U.S. Pat. No. 4,772,675, U.S. Pat. No. 4,871,827, U.S. Pat. No. 4,888,380, U.S. Pat. No. 4,898,910, U.S. Pat. No. 4,906,719 and U.S. Pat. No. 4,962,174, the contents of which are incorporated by reference in the present application.

We may mention, as compound X and/or Y, in particular the polyorganosiloxanes selected from the polymers of formula:

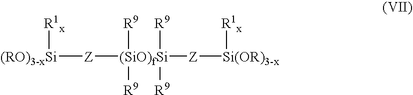

in which R, R¹, R⁹, Z, x and f are as described above.

Compounds X and/or Y can also include a mixture of polymers of formula (VII) above with polymers of the following formula (VIII):

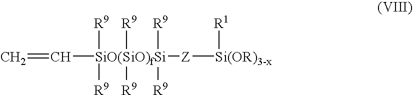

in which R, R¹, R⁹, Z, x, and f are as described above.

When the polyorganosiloxane compound X and/or Y with alkoxysilane group(s) includes said mixture, the various polyorganosiloxanes are present at contents such that the organosilyl terminal chains represent less than 40%, preferably less than 25% in number of terminal chains.

Polyorganosiloxane compounds X and/or Y that are particularly preferred are those of formula (VII) described above. Such compounds X and/or Y are described for example in document WO 01/96450.

As stated above, compounds X and Y can be identical or different.

In particular, compounds X and Y can represent a mixture of polydimethylsiloxanes with methoxysilane groups.

According to a variant, one of the 2 reacting compounds X or Y is of silicone character and the other is of organic character. For example, compound X is selected from organic oligomers or polymers or hybrid organic/silicone oligomers or polymers, said polymers or oligomers comprising at least two alkoxysilane groups, and Y is selected from silicone compounds such as the polyorganosiloxanes described above. In particular, the organic oligomers or polymers are selected from the vinylic, (meth)acrylic oligomers or polymers, polyesters, polyamides, polyurethanes and/or polyureas, polyethers, polyolefins, perfluoropolyethers, dendrimers and hyperbranched organic polymers, and mixtures thereof.

According to one embodiment, compound X of organic character or of hybrid organic/silicone character bears at least one polar group, as described above, capable of forming at least one hydrogen bond with the keratin material.

The organic polymers of vinylic or (meth)acrylic character, bearing alkoxysilane side groups, can in particular be obtained by copolymerization of at least one vinylic or (meth)acrylic organic monomer with a (meth)acryloxypropyltrimethoxysilane, a vinyltrimethoxysilane, a vinyltriethoxysilane, an allyltrimethoxysilane etc.

We may mention for example the (meth)acrylic polymers described in the document of Kusabe, M, Pitture e Vemiei—European Coating; 12-B, pages 43-49, 2005, and notably the polyacrylates with alkoxysilane groups with the designation MAX from Kaneka or those described in the work by Probster, M, Adhesion-Kleben & Dichten, 2004, 481 (1-2), pages 12-14.

The organic polymers resulting from a polycondensation or a polyaddition, such as polyesters, polyamides, polyurethanes and/or polyureas, polyethers, and bearing alkoxysilane side and/or end groups, can result for example from reaction of an oligomeric prepolymer as described above with one of the following silane reaction partners bearing at least one alkoxysilane group: aminopropyltrimethoxysilane, aminopropyltriethoxysilane, aminoethyl aminopropyl trimethoxysilane, glycidoxypropyltrimethoxysilane, glycidoxypropyltriethoxysilane, epoxycyclohexylethyltrimethoxysilane, mercaptopropyltrimethoxysilane.

Examples of polyethers and polyisobutylenes with alkoxysilane groups are described in the work by KUSABE, M., Pitture e Verniei—European Coating; 12-B, pages 43-49, 2005. As examples of polyurethanes with alkoxysilane end groups, we may mention those described in the document PROBSTER, M., Adhesion-Kleben & Dichten, 2004, 481 (1-2), pages 12-14 or alternatively those described in the document LANDON, S., Pitture e Verniei Vol. 73, No. 11, pages 18-24, 1997 or in the document Huang, Mowo, Pitture e Verniei Vol. 0.5, 2000, pages 61-67, and we may notably mention the polyurethanes with alkoxysilane groups from OSI-WITCO-GE.

As polyorganosiloxane compounds X and/or Y, we may mention the resins of type MQ or MT which themselves bear alkoxysilane and/or silanol end groups, for example the poly(isobutylsilsesquioxane) resins functionalized with silanol groups offered under reference SST-S7C41 (three Si—OH groups) by the company Gelest.

2a—Additional Reactive Compound

The polycondensation reaction can also be carried out in the presence of an additional reactive compound comprising at least two alkoxysilane or silanol groups.

We may mention for example:
one or more organic or mineral particles with alkoxysilane and/or silanol groups on their surface, for example fillers surface-treated with said groups.

2b—Catalyst

The condensation reaction can take place in the presence of a metal-based catalyst which can be present in the organic phase of stage 1. It can be introduced via one or other of the compositions comprising X and/or Y or in a separate composition. The catalyst for use in this type of reaction is preferably a titanium-based catalyst.

We may notably mention the catalysts based on tetraalkoxytitanium of formula:

Ti(OR²)_{y}(OR³)_{4-y},

in which R² is selected from the tertiary alkyl radicals such as tert-butyl, tert-amyl and 2,4-dimethyl-3-pentyl; R³ represents an alkyl radical having from 1 to 6 carbon atoms, preferably a methyl, ethyl, n-propyl, isopropyl, n-butyl, sec-butyl, hexyl group and y is a number in the range from 3 to 4, preferably from 3.4 to 4.

The catalyst can be present in the composition at a content ranging from 0.0001% to 20% by weight relative to the total weight of the composition containing it.

As an example of a combination of compounds X and Y bearing alkoxysilane groups and reacting by condensation, we may mention the combination of the following mixtures A′ and B′ produced by the company Dow Corning:

Mixture A′:

| Ingredient (INCI name) | CAS No. | Contents (%) | Function |
|---|---|---|---|
| Bis- | PMN87176 | 25-45 | Polymer |
| Trimethoxysiloxyethyl | | | |
| Tetramethyldisiloxyethyl | | | |
| Dimethicone (1) | | | |
| Silica Silylate | 68909-20-6 | 5-20 | Filler |
| Disiloxane | 107-46-0 | 30-70 | Solvent |

Mixture B′:

| Ingredient (INCI name) | CAS No. | Contents (%) | Function |
|---|---|---|---|
| Disiloxane | 107-46-0 | 80-99 | Solvent |
| Tetra T Butyl Titanate | — | 1-20 | Catalyst |

It should be noted that compounds X and Y, identical, are combined in mixture A′ (cf. (1))

3/Crosslinking in the Presence of Peroxide:

According to one embodiment, the invention relates to particles of core/shell type having a lipophilic core, characterized in that their shell is formed at least partly by a silicone polymer film which is the product of in situ reaction between at least one compound X and at least one compound Y, at least one of compounds X and Y being a polyorganosiloxane and said reaction being of crosslinking type carried out in the presence of a peroxide.

This reaction is preferably performed by heating to a temperature greater than or equal to 50° C., preferably greater than or equal to 80° C., and up to 120° C.

Compounds X and Y, which may be identical or different, have in this case at least two —CH₃ side groups and/or at least two side chains bearing a —CH₃ group.

Compounds X and Y are preferably polyorganosiloxanes and can be selected for example from the non-volatile linear polydimethylsiloxanes of high molecular weight, having a degree of polymerization above 6 and with at least two —CH₃ side groups attached to the silicon atom and/or at least two side chains bearing a —CH₃ group. We may mention for example the polymers described in the Catalogue “Reactive Silicones” of the company Gelest Inc., Edition 2004, page 6, and notably the copolymers (also called gums) of vinylmethylsiloxane-dimethylsiloxane of molecular weight in the range from 500 000 to 900 000 and notably with viscosity above 2 000 000 cSt.

As peroxides that can be used in the invention, we may mention benzoyl peroxide, 2,4-dichlorobenzoyl peroxide and mixtures thereof.

According to one embodiment, the hydrosilylation reaction in the presence of a catalyst, or the condensation reaction, if applicable in the presence of a catalyst, or alternatively the crosslinking reaction in the presence of a peroxide, between compounds X and Y is accelerated by supply of heat, for example by raising the temperature of the system between 25° C. and 180° C.

In general, regardless of the type of reaction by which compounds X and Y react with one another, the molar percentage of X relative to the total of compounds X and Y, i.e. the ratio X/(X+Y)×100, can vary from 5% to 95%, preferably from 10% to 90%, and more preferably from 20% to 80%.

Similarly, the molar percentage of Y relative to the total of compounds X and Y, i.e. the ratio Y/(X+Y)×100, can vary from 5% to 95%, preferably from 10% to 90%, and more preferably from 20% to 80%.

Compound X can have a weight-average molecular weight (Mw) in the range from 150 to 1 000 000, preferably from 200 to 800 000, more preferably from 200 to 250 000.

Compound Y can have a weight-average molecular weight (Mw) in the range from 200 to 1 000 000, preferably from 300 to 800 000, more preferably from 500 to 250 000.

Compound X can represent from 0.1% to 95% by weight relative to the total weight of the composition containing it, preferably from 1% to 90%, and more preferably from 5% to 80%.

Compound Y can represent from 0.1% to 95% by weight relative to the total weight of the composition containing it, preferably from 1% to 90%, and more preferably from 5% to 80%.

The ratio of compound X to compound Y can be varied so as to adjust the reaction rate and therefore the rate of formation of the film or alternatively so as to adapt the properties of the shell formed (for example its adhesive properties) according to the intended application.

In particular, compounds X and Y can be present at a molar ratio X/Y in the range from 0.05 to 20 and preferably from 0.1 to 10.

Compounds X and Y can advantageously be combined with at least one filler. Thus, the particles according to the invention or compositions comprising them can for example include at least one filler selected from silica or surface-treated silica.

### CORE OF THE PARTICLES ACCORDING TO THE INVENTION

The core of the particles according to the invention is formed from at least one lipophilic compound. It may especially comprise at least one oil, an active agent in oil form or an active agent dissolved in an oily phase.

For the purposes of the present invention, the term “oil” denotes any natural plant or animal, or synthetic, oily substance that is liquid at 40° C., possibly having one or more proven biological activities, and which is insoluble in water (less than 2% by weight at room temperature).

The oils that may be used in the core of the particles according to the invention may be chosen especially from those given below in the definition of the physiologically acceptable medium.

The core may also contain at least one lipophilic active agent other than an oil.

One or more liposoluble active materials, which will be dissolved in a solvent oil, may also be encapsulated.

According to this variant, it is the mixture of at least two or even more components that is encapsulated.

Examples of these liposoluble active principles that may be mentioned include D-α-tocopherol, DL-α-tocopherol, D-α-tocopheryl acetate, DL-α-tocopheryl acetate, ascorbyl palmitate, vitamin F glycerides, D vitamins, vitamin D₂, vitamin D₃, retinol, retinyl esters, retinyl palmitate, retinyl propionate, carotenes, including β-carotene, D-panthenol, farnesol, farnesyl acetate, salicylic acid and derivatives thereof, for instance 5-n-octanoylsalicylic acid, alkyl esters of α-hydroxy acids such as citric acid, lactic acid, glycolic acid, asiatic acid or madecassic acid, asiaticoside, the total extract of Centella asiatica, β-glycyrrhetinic acid, α-bisabolol, ceramides, for instance 2-oleoylamino-1,3-octadecane, phytanetriol, phospholipids of marine origin rich in polyunsaturated essential fatty acids, ethoxyquine, extract of rosemary, extract of lemon balm, quercetin, extract of dried microalgae (algoxan red from Algatec), essential oil of bergamot, octyl methoxycinnamate (Parsol MCX—Givaudan-Roure), butylmethoxydibenzoylmethane (Parsol 1789-Givaudan-Roure), octyl triazone (Uvinul T150-BASF) and 3,5-di-tert-butyl-4-hydroxy-3-benzylidenecamphor.

Moreover, any molecule with a therapeutic effect used in the pharmaceutical field, provided that it has an oily nature and/or sufficient solubility in the oil under consideration, may also be encapsulated. These molecules may thus be steroidal or non-steroidal anti-inflammatory drugs, antifungal agents, antibacterial agents, antibiotics, antimitotic agents, anaesthetics, analgesics, antiseptics and antiviral agents.

However, they may also be liposoluble compounds that are active in other fields, for instance pesticides or herbicides, or a mixture of the abovementioned active agents.

Needless to say, the content of oil(s) and, where appropriate, of liposoluble active material(s) (oil or oil+active agent) dissolved in the solvent depends on the solubility thereof.

### Process for Preparing the Particles According to the Invention

As stated previously, the particles according to the invention may be obtained according to conventional techniques based either on the principle of emulsification of a sparingly water-miscible organic phase in an aqueous phase, or on that of solvent tipping, which consists in placing a water-miscible organic phase in contact with an aqueous phase. After these two protocols, the particles formed may be recovered by evaporation of the organic solvent.

These two techniques are described in particular, respectively, in documents FR 2 864 900 and EP 274 961.

During the placing of the two phases in contact, according to one or the other of the two abovementioned techniques, there is formation of droplets and in situ reaction between compounds X and Y, or even the additional reactive compound(s) if present, and thus formation of the expected particles.

As regards the first variant based on the formation of an emulsion of organic phase in water, it uses a standard emulsification tool. The size of the drops of the emulsion formed by placing the organic phase in contact with the aqueous phase may be refined by various known means, for instance high-pressure homogenization or ultrasonication. Finally, the organic solvent is evaporated off. During this evaporation, compounds X and Y become concentrated in the organic phase and the reaction takes place between these two compounds in the presence, where appropriate, of a catalyst or a peroxide if necessary for their interaction to form the shell of the particles.

As regards the second variant, it involves the dissolution of the lipophilic compound and of compounds X and Y intended to form the polymer that is to constitute the shell of the particle, in an organic solvent in the presence, where appropriate, of a catalyst or of a peroxide if necessary for their interaction. The formation of the particles is initiated by adding the aqueous phase, with stirring, to the organic phase. A spontaneous emulsion forms when the aqueous phase diffuses into the organic phase. Compounds X and Y then react together to form the silicone polymer film that constitutes the shell of the particles. The organic solvent(s) is (are) then evaporated off.

A Organic Phase

Whatever the variant considered, the organic phase is formed from one or more mutually miscible organic solvents whose boiling point is lower than that of water and thus generally less than 100° C. at atmospheric pressure.

In the case of the first variant, the organic phase is sparingly water-miscible.

For the purposes of the invention, the expression “sparingly water-miscible” means that the organic solvent under consideration has a miscibility with water of less than 10% by weight at room temperature.

As examples of solvents that are suitable for the invention, mention may be made in particular of ethyl acetate, butyl acetate, dichloromethane, cyclohexane, heptane, 1-chlorobutane, chloroform and ethyl formate, and mixtures thereof.

On the other hand, in the case of the second variant, the organic phase is water-miscible. As examples of solvents that are suitable for this second variant, mention may be made of acetone and also lower alcohols such as ethanol, isopropanol and methanol.

Whatever the variant considered, the organic phase contains at least compounds X and Y in dissolved form and one or more lipophilic compounds to be encapsulated.

Besides these compounds, the organic phase may comprise an additional reactive compound as described previously, a catalyst or a peroxide if necessary for the reaction between compounds X and Y, and optionally a compound capable of forming a lamellar phase at the surface of the expected particles.

In all cases, compounds X and Y, which are diluted in the organic phase, do not react together.

B Aqueous Phase

For its part, the aqueous phase advantageously contains at least one surfactant in order, firstly, to facilitate the formation of the emulsion that is formed during the placing in contact of the two phases and, secondly, to stabilize it.

This surfactant or this mixture of surfactants is generally present in an amount of from 0.05% to 25% and especially from 1% to 20% by weight relative to the weight of the organic solvent medium to be dispersed. Its HLB (hydrophilic-lipophilic balance) is generally adjusted so as to be favourable for the formation of emulsions of oil-in-water type.

The following nonionic surfactants are especially suitable for use in the invention:
alkyl esters or ethers of glycerol or of polyglycerol consisting of from 1 to 10 glycerol “units” and of at least one alkyl chain (acid for the esters and alcohol for the ethers) containing from 12 to 22 carbon atoms. It may be saturated or unsaturated, and branched or unbranched. Examples that may be mentioned include Nikkol DGMS® (diglyceryl monostearate), Nikkol decaglyn 2IS® (decaglyceryl diisostearate) and triglyceryl hexadecyl ether,
mixed esters of fatty acids or of fatty alcohols, of carboxylic acids and of glyceryl, chosen, for example, from mixed esters of C₈-C₂₂ fatty acid or fatty alcohol and of α-hydroxy acid and/or of succinic acid with glycerol, and mixtures thereof. By way of example, mention may be made of the mixed ester of glycerol and of the mixture of citric acid, lactic acid, linoleic acid and oleic acid (CTFA name: Glyceryl citrate/lactate/linoleate/oleate) sold by the company Hüls under the name Imwitor 375®; the mixed ester of succinic acid and of isostearyl alcohol with glycerol (CTFA name: Isostearyl diglyceryl succinate) sold by the company Hüls under the name Imwitor 780 K the mixed ester of citric acid and of stearic acid with glycerol (CTFA name: Glyceryl stearyl citrate) sold by the company Hüls under the name Imwitor 370®; the mixed ester of lactic acid and of stearic acid with glycerol (CTFA name: Glyceryl stearate lactate) sold by the company Danisco under the name Lactodan B30® or Rylo LA30®,
ethoxylated fatty ethers or ethoxylated fatty esters comprising from 2 to 50 ethylene oxide units and at least one alkyl chain (acid for the esters and alcohol for the ethers) containing from 12 to 22 carbon atoms. It may be saturated or unsaturated, and branched or unbranched. Examples that will be mentioned include the Brij® series (ethoxylated fatty alcohols) sold by the company Uniqema, the Myrj® series (ethoxylated stearates) sold by the company Uniqema, and PEG 400 isostearate, also sold by Uniqema,
oxyethylenated or non-oxyethylenated fatty esters of sorbitan. They comprise at least one sorbitan unit and, when they are oxyethylenated, from 2 to 50 ethylene oxide units, and at least one alkyl chain (fatty acid) containing from 12 to 22 carbon atoms. It may be saturated or unsaturated, and branched or unbranched. Examples that will be mentioned include the Span® series (sorbitan esters) and the Tween® series (oxyethylenated sorbitan esters) sold by Uniqema,
sugar fatty esters or sugar fatty ethers. The surfactant used is chosen, for example, from C₈-C₂₂ fatty acid esters of sucrose, maltose, glucose and fructose, C₁₄-C₂₂ fatty acid esters of methylglucose, alkylpolyglucosides, and mixtures thereof. The alkyl chain(s) may be saturated or unsaturated, and branched or unbranched.

The C₈-C₂₂ or C₁₄-C₂₂ fatty acids forming the fatty unit of the esters that may be used in the composition of the invention comprise at least one saturated or unsaturated linear alkyl chain, of 8 to 22 or of 14 to 22 carbon atoms, respectively. The fatty unit of the esters may be chosen especially from stearates, behenates, arachidonates, palmitates, myristates, laurates and caprates, and mixtures thereof. Stearates are especially used.

Examples of esters or mixtures of esters of fatty acid and of sucrose, maltose, glucose or fructose that may be mentioned include sucrose monostearate, sucrose distearate and sucrose tristearate, and mixtures thereof, such as the products sold by the company Croda under the name Crodesta® F50, F70, F110 and F160, respectively having an HLB (Hydrophilic-Lipophilic Balance) of 5, 7, 11 and 16; and an example of esters or mixtures of esters of fatty acid and of methylglucose that may be mentioned is methylglucose polyglyceryl-3 distearate, sold by the company Goldschmidt under the name Tegocare 450®. Mention may also be made of monoesters of glucose or of maltose such as methyl O-hexadecanoyl-6-D-glucoside and O-hexadecanoyl-6-D-maltoside.

The sugar fatty alcohol ethers that may be used as surfactants in the composition according to the invention may be chosen especially from the group comprising ethers or mixtures of ethers of C₈-C₂₂ fatty alcohol and of glucose, maltose, sucrose or fructose, and ethers or mixtures of ethers of C₁₄-C₂₂ fatty alcohol and of methylglucose. They are especially alkylpolyglucosides.

The C₈-C₂₂ or C₁₄-C₂₂ fatty alcohols forming the fatty unit of the ethers that may be used according to the invention comprise a saturated or unsaturated linear alkyl chain containing, respectively, from 8 to 22 or from 14 to 22 carbon atoms. The fatty unit of the ethers may be chosen especially from decyl, cetyl, behenyl, arachidyl, stearyl, palmityl, myristyl, lauryl, capryl and hexadecanoyl, and mixtures thereof such as cetearyl.

Examples of sugar fatty alcohol ethers that may be mentioned include alkylpolyglucosides such as decyl glucoside and lauryl glucoside sold, for example, by the company Henkel under the respective names Plantaren 2000® and Plantaren 1200®, cetostearyl glucoside optionally as a mixture with cetostearyl alcohol, sold, for example, under the name Montanov 68® by the company SEPPIC, under the name Tegocare CG90® by the company Goldschmidt and under the name Emulgade KE3302® by the company Henkel, and also arachidyl glucoside, for example in the form of the mixture of arachidyl and behenyl alcohols and of arachidyl glucoside, sold under the name Montanov 202® by the company SEPPIC,
block copolymers of ethylene oxide and of propylene oxide. The block copolymers of ethylene oxide and of propylene oxide that may be used as surfactants in the compositions according to the invention may be chosen especially from the block copolymers of formula (A):

HO(C₂H₄O)ₓ(C₃H₆O)_{y}(C₂H₄O)_{z}H (A)

in which x, y and z are integers such that x+z may range from 2 to 280 and may preferably range from 14 to 100. These polymers are sold especially under the name Pluronic® or Lutrol® by BASF, or Symperonic® by Uniqema,
hydrogenated or non-hydrogenated soybean or egg lecithins, optionally enriched in phosphatidylcholine,
silicone surfactants comprising at least one oxyethylene or oxypropylene chain. Examples that may be mentioned include those described in patents U.S. Pat. No. 5,364,633 and U.S. Pat. No. 5,411,744, and in particular a compound of formula (I):

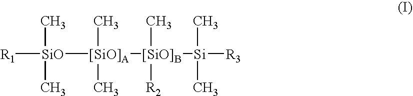

in which:
R₁, R₂ and R₃, independently of each other, represent a C₁-C₆ alkyl radical or a radical —(CH₂)ₓ—(OCH₂CH₂)_{y}—(OCH₂CH₂CH₂)_{z}—OR₄, at least one radical R₁, R₂ or R₃ not being an alkyl radical; R₄ being a hydrogen, an alkyl radical or an acyl radical;
A is an integer ranging from 0 to 200;
B is an integer ranging from 0 to 50; with the condition that A and B are not simultaneously equal to zero;
x is an integer ranging from 1 to 6;
y is an integer ranging from 1 to 30;
z is an integer ranging from 0 to 5.

According to one particular embodiment, in the compound of formula (I), the alkyl radical is a methyl radical, x is an integer ranging from 2 to 6 and y is an integer ranging from 4 to 30.

Examples of silicone surfactants of formula (I) that may be mentioned include the compounds of formula (II):

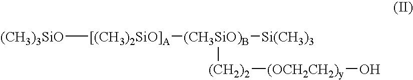

in which A is an integer ranging from 20 to 105, B is an integer ranging from 2 to 10 and y is an integer ranging from 10 to 20.

Examples of silicone surfactants of formula (I) that may also be mentioned include the compounds of formula (III):

HO—(CH₂CH₂O)_{y}—(CH₂)₃-[(CH₃)₂SiO]_{A′}—(CH₂)₃—(OCH₂CH₂)_{y}—OH (III)

in which A′ and y are integers ranging from 10 to 20.

Compounds of the invention that may be used include those sold by the company Dow Corning under the names DC 5329, DC 7439-146, DC2-5695 and Q4-3667. The compounds DC 5329, DC 7439-146 and DC2-5695 are compounds of formula (II) in which, respectively, A is 22, B is 2 and y is 12; A is 103, B is 10 and y is 12; A is 27, B is 3 and y is 12. The compound Q4-3667 is a compound of formula (III) in which A is 15 and y is 13. But also:
alkyl ether citrates,
alkoxylated alkenyl succinates,
alkoxylated glucose alkenyl succinates,
alkoxylated methyl glucose alkenyl succinates.

These surfactants may be used alone or in combination. Their content, relative to the encapsulated oily phase (after evaporating off the solvent) may be between 0.1% and 30% by weight.

In order to improve the stability of this emulsion, or even to slightly further reduce the size of the drops of the organic solvent medium, it may be advantageous to add from 0.01% to 5% by weight, relative to the total weight of the dispersion, of at least one water-soluble ionic surfactant with an HLB value of greater than 11. This type of ionic surfactant appears to generate an electric charge at the surface of the nanocapsules and thus promotes the manifestation of electrostatic repulsions between them. This or these ionic, anionic or cationic surfactant(s) may be chosen from:
anionic amphiphilic lipids, for instance:
alkali metal salts of dicetyl and dimyristyl phosphate,
alkali metal salts of cholesteryl sulfate,
alkali metal salts of cholesteryl phosphate,
lipoamino acids and salts thereof, such as monosodium and disodium acylglutamates, for instance the disodium salt of N-stearoyl-L-glutamic acid sold under the name Amisoft HS21P by the company Ajinomoto,
the sodium salts of phosphatidic acid,
phospholipids,
alkylsulfonic derivatives especially of formula:

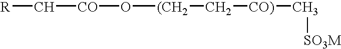

in which R represents C₁₆-C₂₂ alkyl radicals, in particular C₁₆H₃₃ and C₁₈H₃₇ radicals, taken as a mixture or separately, and M is an alkali metal or alkaline-earth metal such as sodium,
cationic amphiphilic lipids of quaternary ammonium salt type, and fatty amines and salts thereof, for instance:
the quaternary ammonium salts of general formula (IV) below:

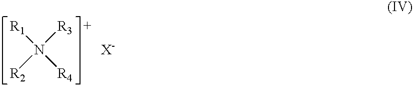

in which the radicals R₁, R₂, R₃ and R₄, which may be identical or different, represent a linear or branched aliphatic radical containing from 1 to 30 carbon atoms or an aromatic radical such as aryl or alkylaryl. The aliphatic radicals may comprise heteroatoms especially such as oxygen, nitrogen, sulfur and halogens. The aliphatic radicals are chosen, for example, from alkyl, alkoxy, polyoxy(C₂-C₆)alkylene, alkylamide, (C₁₂-C₂₂)alkylamido(C₂-C₆)alkyl, (C₁₂-C₂₂)alkylacetate and hydroxyalkyl, containing from about 1 to 30 carbon atoms; X is an anion chosen from the group of halides, phosphates, acetates, lactates, (C₂-C₆)alkyl sulfates, and alkyl- or alkylarylsulfonates. Quaternary ammonium salts of formula (IV) that are preferred include, firstly, tetraalkylammonium chlorides, for instance dialkyldimethylammonium or alkyltrimethylammonium chlorides, in which the alkyl radical contains from about 12 to 22 carbon atoms, in particular behenyltrimethylammonium, distearyldimethylammonium, cetyltrimethylammonium or benzyldimethylstearylammonium chloride, or alternatively, secondly, stearamidopropyl-dimethyl(myristyl acetate)ammonium chloride sold under the name “Ceraphyl 70®” by the company Van Dyk,
quaternary ammonium salts of imidazolinium, for instance those of formula (V) below:

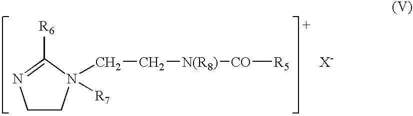

in which R₅ represents an alkenyl or alkyl radical containing from 8 to 30 carbon atoms, for example fatty acid derivatives of tallow; R₆ represents a hydrogen atom, an alkyl radical containing from 1 to 4 carbon atoms or an alkenyl or alkyl radical containing from 8 to 30 carbon atoms; R₇ represents an alkyl radical containing from 1 to 4 carbon atoms; R₈ represents a hydrogen atom or an alkyl radical containing from 1 to 4 carbon atoms; X is an anion chosen from the group of halides, phosphates, acetates, lactates, alkyl sulfates, and alkyl- or alkylarylsulfonates. Preferably, R₅ and R₆ denote a mixture of alkenyl or alkyl radicals containing from 12 to 21 carbon atoms, for example fatty acid derivatives of tallow, R₇ denotes a methyl radical, R₈ denotes hydrogen. Such a product is sold, for example, under the name “Rewoquat W 75” by the company Rewo,
the diquaternary ammonium salts of formula (VI) below:

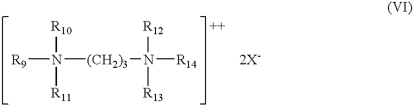

in which R₉ denotes an aliphatic radical containing from about 16 to 30 carbon atoms; R₁₀, R₁₁, R₁₂, R₁₃ and R₁₄ are chosen from hydrogen and an alkyl radical containing from 1 to 4 carbon atoms; and X is an anion chosen from the group of halides, acetates, phosphates, nitrates and methyl sulfates. Such diquaternary ammonium salts especially include propane tallow diammonium dichloride.

The content of ionic surfactant, when it is combined with the nonionic surfactant(s) present in the aqueous medium, is generally adjusted so as to represent from 2% to 100% by weight relative to the weight of the medium.

C Compound Capable of Forming a Lamellar Phase

It is often desirable or necessary to provide particles, for example microparticles or nanocapsules, with a “lamellar” coating. This is a structure organized as one or more lipid leaflets each constituted of a bilayer of amphiphilic molecules similar to that of biological membranes. The polymer envelope of the particles, for example the microparticles or the nanocapsules according to the invention, may thus be surrounded by a lamellar coating having a structure organized as one or more leaflets each constituted of a double layer of amphiphilic molecules constituting a coating agent. Besides its function of adjusting the size of the particles, for example the microparticles or the nanocapsules, this coating improves the leaktightness of the particles, for example the microparticles or the nanocapsules, with respect to leakage of the active agent into another lipid phase of the composition.

The term “lamellar phase” (phase D according to Ekwall) means a liquid crystal phase of plane symmetry, comprising several bilayers of amphiphilic molecules arranged in parallel and separated by a liquid medium that is generally water.

A more precise definition of this term is given in Ekwall (1968), Adv. Liq. Cryst. (Brown G. H., ed.), chap. 1, 14. This phase has a characteristic texture under a polarized-light microscope, a more precise description of which may be found in Roservear (1968), JSCC, 19, 581 and in Lachampt and Vila (1969), Revue Française des Corps Gras, No 2, 87-111.

The lamellar coating is obtained with surfactants of hydrophobic nature, which are soluble in the organic phase used in the process described above and which are capable, in the presence of water, of forming the lipid double layers described above. In the encapsulation process used by the inventors, the coating surfactant is dissolved in the organic phase containing compounds X and Y and the lipid phase.

Examples of such coating surfactants that may be mentioned include phospholipids such as lecithin, as described in document EP-A-447 318; certain polycondensates of ethylene oxide and of propylene oxide, for instance the products sold under the name Pluronic® by the company BASF, such as Pluronic® L121, or under the name Synperonic® by the company ICI; or silicone surfactants (silicones comprising at least one oxyethylene and/or oxypropylene chain) capable of forming lamellar structures, such as those described in documents U.S. Pat. No. 5,364,633 and U.S. Pat. No. 5,411,744 used in patent application FR-A-2 742 677, for example those sold by the company Dow Corning under the names DC 5329, DC 7439-146, DC2-5695 and Q4-3667; and mixtures thereof.

The particles, for example the nanocapsules or microcapsules, obtained may be introduced into any type of galenical formulation, on condition that they ensure the stability thereof, such as gels, oil/water, water/oil or multiple (W/O/W or O/W/O) emulsions, sera, lotions, etc. From 0.5% to 60% of particle suspension according to the invention may be introduced into the final formulation.

They may especially be used as vehicle for the lipophilic active agent in cosmetic or therapeutic compositions.

As stated previously, a composition in accordance with the invention comprises a physiologically acceptable medium.

The expression “physiologically acceptable medium” is intended to denote a medium that is particularly suitable for applying a composition according to the invention to the skin, the lips, the eyelashes, the eyebrows or the nails. The physiologically acceptable medium is generally suited to the nature of the support onto which the composition is to be applied, and also to the aspect in which the composition is intended to be conditioned.

The physiologically acceptable medium may comprise an aqueous phase, essentially comprising water.

It may also comprise a mixture of water and of water-miscible solvent (miscibility with water of greater than 50% by weight at 25° C.), for instance one, or a mixture of, lower monoalcohol(s) containing from 1 to 5 carbon atoms, such as ethanol or isopropanol, glycols containing from 2 to 8 carbon atoms, such as propylene glycol, ethylene glycol, 1,3-butylene glycol or dipropylene glycol, C₃-C₄ ketones and C₂-C₄ aldehydes, and mixtures thereof.

The physiologically acceptable medium may also comprise a liquid fatty phase, comprising one or more volatile or non-volatile oils, and/or a solid fatty phase comprising one or more waxes and/or pasty compounds, and a mixture thereof.

For the purposes of the patent application, the term “liquid fatty phase” means a fatty phase that is liquid at room temperature (25° C.) and atmospheric pressure (760 mmHg), composed of one or more non-aqueous fatty substances that are liquid at room temperature, also known as oils or organic solvents.

The oil may be chosen from volatile oils and/or non-volatile oils, and mixtures thereof.

The oil(s) may be present in a content ranging from 1% to 90% by weight and preferably from 5% to 50% by weight relative to the total weight of the composition.

These oils may be hydrocarbon-based oils, silicone oils or fluoro oils, or mixtures thereof.

The term “hydrocarbon-based oil” means an oil mainly containing hydrogen and carbon atoms and possibly oxygen, nitrogen, sulfur and phosphorus atoms. The hydrocarbon-based oils may be chosen from hydrocarbon-based oils containing from 8 to 16 carbon atoms, and especially branched C₈-C₁₆ alkanes, for instance C₈-C₁₆ isoalkanes of petroleum origin (also known as isoparaffins), for instance isododecane (also known as 2,2,4,4,6-pentamethylheptane), isodecane, isohexadecane and, for example, the oils sold under the trade names Isopar® and Permethyl®, C₈-C₁₆ branched esters and isohexyl neopentanoate, and mixtures thereof.

Hydrocarbon-based oils that may also be mentioned include:
hydrocarbon-based oils of plant origin, such as triesters of fatty acids and of glycerol, the fatty acids of which may have varied chain lengths from C4 to C24, these chains possibly being linear or branched, and saturated or unsaturated; these oils are especially wheatgerm oil, sunflower oil, grapeseed oil, sesame seed oil, corn oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppyseed oil, pumpkin oil, marrow oil, blackcurrant oil, evening primrose oil, millet oil, barley oil, quinoa oil, rye oil, safflower oil, candlenut oil, passionflower oil or musk rose oil; or caprylic/capric acid triglycerides, for instance those sold by the company Stéarineries Dubois or those sold under the names Miglyol 810, 812 and 818 by the company Dynamit Nobel;
synthetic ethers containing from 10 to 40 carbon atoms;
apolar hydrocarbon-based oils, for instance squalene, linear or branched hydrocarbons such as liquid paraffin, liquid petroleum jelly and naphthalene oil, hydrogenated or partially hydrogenated polyisobutene, isoeicosane, squalane, decene/butene copolymers and polybutene/polyisobutene copolymers, especially Indopol L-14, and polydecenes such as Puresyn 10, and mixtures thereof;
synthetic esters, for instance oils of formula R₁COOR₂ in which R₁ represents a linear or branched fatty acid residue containing from 1 to 40 carbon atoms and R₂ represents a hydrocarbon-based chain, which is especially branched, containing from 1 to 40 carbon atoms, on condition that R₁+R₂≧10, for instance Purcellin oil (cetostearyl octanoate), isopropyl myristate, isopropyl palmitate, C₁₂ to C₁₅ alkyl benzoates, hexyl laurate, diisopropyl adipate, isononyl isononanoate, 2-ethylhexyl palmitate, isostearyl isostearate, alcohol or polyalcohol octanoates, decanoates or ricinoleates, for instance propylene glycol dioctanoate; hydroxylated esters, for instance isostearyl lactate or diisostearyl malate; and pentaerythritol esters;
fatty alcohols that are liquid at room temperature with a branched and/or unsaturated carbon-based chain containing from 12 to 26 carbon atoms, for instance octyldodecanol, isostearyl alcohol, oleyl alcohol, 2-hexyldecanol, 2-butyloctanol or 2-undecylpentadecanol;
higher fatty acids such as oleic acid, linoleic acid or linolenic acid;
and mixtures thereof.

As silicone oils that may be used in the invention, mention may be made especially of octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyl-tetrasiloxane and dodecamethylpentasiloxane, and mixtures thereof.

The silicone oils may also be:
polydimethylsiloxanes (PDMS),
polydimethylsiloxanes comprising alkyl or alkoxy groups, which are pendent and/or at the end of a silicone chain, these groups each containing from 3 to 40 carbon atoms,
phenyl silicones, for instance phenyl trimethicones, phenyl dimethicones, phenyltrimethylsiloxydiphenylsiloxanes, diphenyl dimethicones, diphenylmethyl-diphenyltrisiloxanes and 2-phenylethyl trimethylsiloxysilicates;
and mixtures thereof.

The composition may comprise from 0.1% to 80% by weight, preferably from 1% to 60% by weight, better still from 5% to 50% by weight and even better still from 14% to 40% by weight of non-volatile oil(s) relative to its total weight.

The compositions according to the invention may also comprise at least one fatty substance that is solid at room temperature, especially chosen from waxes and pasty fatty substances, and mixtures thereof. These fatty substances may be of animal, plant, mineral or synthetic origin.

The compositions may comprise at least one dyestuff chosen, for example, from pigments, nacres, colorants and materials with an effect, and mixtures thereof.

The term “colorants” should be understood as meaning compounds, generally organic compounds, which are soluble in at least one oil or in an aqueous-alcoholic phase.

The term “pigments” should be understood as meaning white or coloured, mineral or organic particles, which are insoluble in an aqueous medium, and which are intended to colour and/or opacify the resulting film.

The term “nacres”, or nacreous pigments, should be understood as meaning iridescent or non-iridescent coloured particles of any form, especially produced by certain molluscs in their shell or else synthesized, and which have a colour effect via optical interference.

The pigment may also be a lake. The term “lake” means insolubilized colorants adsorbed onto insoluble particles, the assembly thus obtained remaining insoluble during use.

The cosmetic compositions according to the invention may also comprise at least one filler, especially in a content ranging from 0.01% to 50% by weight and preferably ranging from 0.01% to 30% by weight relative to the total weight of the composition.

The fillers may be mineral or organic, of any form, platelet-shaped, spherical or oblong, irrespective of the crystallographic form (for example lamellar, cubic, hexagonal, orthorhombic, etc.). Mention may be made of talc, mica, silica, silica surface-treated with a hydrophobic agent, kaolin, polyamide (Nylon®) powder (Orgasol® from Atochem), poly-β-alanine powder and polyethylene powder, tetrafluoroethylene polymer (Teflon®) powders, lauroyllysine, starch, boron nitride, hollow polymer microspheres such as those of polyvinylidene chloride/acrylonitrile, for instance Expancel® (Nobel Industrie), or of acrylic acid copolymers (Polytrap® from the company Dow Corning) and silicone resin microbeads (for example Tospearls® from Toshiba), elastomeric polyorganosiloxane particles, precipitated calcium carbonate, magnesium carbonate, magnesium hydrogen carbonate, hydroxyapatite, hollow silica microspheres (Silica Beads® from Maprecos), glass or ceramic microcapsules, and metal soaps derived from organic carboxylic acids containing from 8 to 22 carbon atoms and preferably from 12 to 18 carbon atoms, for example zinc stearate, magnesium stearate, lithium stearate, zinc laurate or magnesium myristate.

The compositions according to the invention may also contain ingredients commonly used in cosmetics, such as vitamins, thickeners, film-forming agents, gelling agents, trace elements, softeners, sequestrants, fragrances, basifying agents, acidifying agents, preserving agents, sunscreens, surfactants, antioxidants, fibres or care active agents, or mixtures thereof.

The cosmetic compositions according to the invention may be compositions for making up or caring for keratin materials, in particular for the skin, the lips, the eyelashes, the eyebrows or the nails.

The care compositions may be in the form of a protective, treatment or care composition for the face, for the hands, for the feet, for the major anatomical folds or for the body (for example a day cream, a night cream, a makeup-removing cream, an antisun composition, a protective or care body milk, an after-sun milk, a skincare lotion, gel or mousse, or an artificial tanning composition); an aftershave composition.

They may also be compositions more particularly intended for makeup, for instance a foundation, a concealer product, a lip balm, a lip gloss, a mascara, a body makeup product or a skin colouring product.

The examples and figures submitted below are given as non-limiting illustrations of the invention.

### FIGURE

FIG. 1 presents the results of a leakage test performed with the nanocapsules in accordance with the invention of Example 2 in comparison with the control nanocapsules of Example 3.

In the examples of compositions described hereunder, the combination of the following mixtures A and B produced by the company Dow Corning is used as compounds X and Y:

MIXTURE A:

| Ingredient (INCI name) | CAS No. | Contents (%) | Function |
|---|---|---|---|
| Dimethyl Siloxane, | 68083-19-2 | 55-95 | Polymer |
| Dimethylvinylsiloxy- | | | |
| terminal | | | |
| Silica Silylate | 68909-20-6 | 10-40 | Filler |
| 1,3-Diethenyl-1,1,3,3- | 68478-92-2 | Trace | Catalyst |
| Tetramethyldisiloxane | | | |
| complexes | | | |
| Tetramethyldivinyldisiloxane | 2627-95-4 | 0.1-1 | Polymer |

MIXTURE B:

| Ingredient (INCI name) | CAS No. | Contents (%) | Function |
|---|---|---|---|
| Dimethyl Siloxane, | 68083-19-2 | 55-95 | Polymer |
| Dimethylvinylsiloxy- | | | |
| terminal | | | |
| Silica Silylate | 68909-20-6 | 10-40 | Filler |
| Dimethyl, | 68037-59-2 | 1-10 | Polymer |
| Methylhydrogen | | | |
| Siloxane, trimethylsiloxy- | | | |
| terminal | | | |

### EXAMPLE 1

### Vitamin E Acetate Nanocapsules in Accordance with the Invention

Organic phase:

| | | |
|---|---|---|
| Tocopheryl acetate | 15 | g |
| Mixture A | 1 | g |
| Mixture B | 1 | g |
| Dichloromethane | 66.7 | ml |

Aqueous phase:

| | | |
|---|---|---|
| Disodium N-stearoyl-L-glutamic acid (Amisoft HS21P from | 0.5 | g |
| Ajinomoto) | | |
| Distilled water | 82.5 | g |

The constituents of the aqueous phase are introduced into a beaker, with stirring, at 40° C. This aqueous phase is then cooled to room temperature.

The constituents of the organic phase are dissolved, with stirring, at room temperature in another beaker.

An emulsion is prepared by pouring the organic phase into the aqueous phase and stirring using a stirrer of rotor-stator type, for instance an Ultra-Turrax blender.

The size of the drops is then refined by ultrasonication.

The dichloromethane and part of the water are evaporated off using a rotary evaporator until a final mass of 100 g is obtained.

A suspension of nanocapsules having a mean diameter of 256 nm, measured by dynamic light scattering on a BI90+ machine from Brookhaven Instruments, is obtained.

### EXAMPLE 2

### Caprylic/Capric Triglycerides Nanocapsules Containing a Marker, in Accordance with the Invention

Organic phase:

| | | |
|---|---|---|
| Caprylic/capric triglycerides (Myritol 318 from Cognis) | 15 | g |
| Mixture A | 1.5 | g |
| Mixture B | 1.5 | g |
| 2,5-Thiophenediylbis(5-tert-butyl-1,3-benzoxazole) (Uvitex | 0.15 | g |
| OB from Ciba) | | |
| Dichloromethane | 66.7 | ml |

Aqueous phase:

| | | |
|---|---|---|
| Disodium N-stearoyl-L-glutamic acid (Amisoft HS21P from | 0.5 | g |
| Ajinomoto) | | |
| Distilled water | 81.35 | g |

The procedure is the same as that described in Example 1.

A suspension of nanocapsules having a mean diameter of 262 nm, measured by dynamic light scattering on a BI90+ machine from Brookhaven Instruments, is obtained.

### EXAMPLE 3

### Caprylic/Capric Triglycerides Nanocapsules Containing a Marker, Not in Accordance with the Invention Since they are Based on Preformed Silicone Polymer

Organic phase:

| | | |
|---|---|---|
| Caprylic/capric triglycerides (Myritol 318 from Cognis) | 2.5 | g |
| Polymethylsilsesquioxane (Belsil PMS MK from Wacker) | 0.5 | g |
| Dimethicone copolyol (DC SH 3773 M from Dow Corning) | 0.5 | g |
| 2,5-Thiophenediylbis(5-tert-butyl-1,3-benzoxazole) (Uvitex | 0.025 | g |
| OB from Ciba) | | |
| Acetone | 100 | ml |

Aqueous phase:

| | | |
|---|---|---|
| Condensate of ethylene oxide and propylene oxide and | 0.25 | g |
| ethylene oxide (128 EO/54 PO/128 EO) (Synperonic PE/F108 | | |
| from Uniqema) | | |
| Distilled water | 200 | g |

The various constituents of the organic phase are dissolved in a beaker, with stirring, at room temperature. The constituents of the aqueous phase are dissolved, with stirring, at room temperature in another beaker.

The organic phase is poured into the aqueous phase with continued stirring.

The acetone and part of the water are then evaporated off using a rotary evaporator until a final volume of 50 mL is obtained.

A suspension of nanocapsules having a mean diameter of 294 nm, measured by dynamic light scattering on a BI90+ machine from Brookhaven Instruments, is obtained.

### EXAMPLE 4

### Comparison of the Nanocapsules of Examples 2 and 3 in Terms of Leakage of the Encapsulated Marker

The suspension of nanocapsules obtained in Example 2 is diluted threefold with an aqueous 0.5% disodium N-stearoyl-L-glutamic acid solution (Amisoft HS21P from Ajinomoto). A suspension of nanocapsules having the following composition is thus obtained:

| | | |
|---|---|---|
| Caprylic/capric triglycerides (Myritol 318 from Cognis) | 5 | g |
| Mixture A | 0.5 | g |
| Mixture B | 0.5 | g |
| 2,5-Thiophenediylbis(5-tert-butyl-1,3-benzoxazole) | 0.05 | g |
| (Uvitex OB from Ciba) | | |
| Disodium N-stearoyl-L-glutamic acid solution | 0.5 | g |
| (Amisoft HS21P from Ajinomoto) | | |
| Distilled water | 93.45 | g |

These nanocapsules are compared with those obtained in Example 3, which also contain 5% of encapsulated oil and 0.05% of marker (Uvitex OB), according to the following leakage test:

40 g of nanocapsule suspension and 40 g of capric/caprylic triglycerides, which will be referred to as the “extracting oily phase”, are placed in a flask.

These two phases are stirred so as to form a coarse emulsion of the extracting oily phase in the solution of nanocapsules.

At a time T, 5 mL of the mixture are taken. These 5 mL are centrifuged at 50 000 rpm for 10 minutes in order to separate the extracting oily phase from the nanocapsule suspension.

Confirmation was made beforehand that this centrifugation does not deteriorate the nanocapsules and therefore does not cause any additional leakage.

The supernatant extracting oily phase is taken up and diluted with acetone.

The absorbance of the solution obtained is measured at 392 nm, which makes it possible to obtain the concentration of Uvitex OB that has transferred from the core of the nanocapsules into the extracting oily phase at time T.

This test is performed in parallel for the two nanocapsule suspensions.

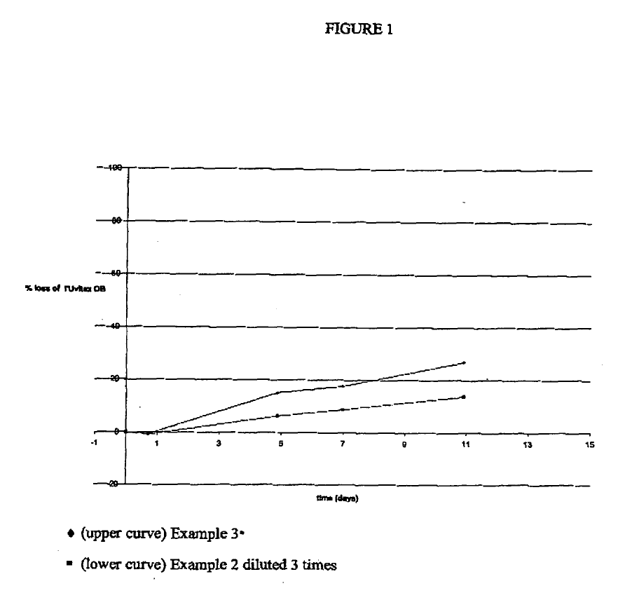

The results are illustrated in FIG. 1.

It is found that the leakage is lower for the nanocapsules whose shell is formed by the reaction of compounds A and B under consideration according to the invention than for a polymer of polymethylsilsesquioxane type. After 11 days, the leakage is 14%, as opposed to 27%. The leakage is virtually divided by a factor of 2.

Although the present invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. Particle of core/shell type having a lipophilic core, wherein its shell is formed at least partly by a silicone polymer film which is the product of in situ reaction between at least one compound X and at least one compound Y, at least one of compounds X and Y being a polyorganosiloxane and said reaction being of hydrosilylation type carried out in the presence of a catalyst, condensation type carried out, if applicable, in the presence of a catalyst, or crosslinking type carried out in the presence of a peroxide.

2. Particle according to claim 1, wherein the core comprises at least one oil and/or a lipophilic cosmetic or therapeutic active agent.

3. Particle according to claim 1, wherein the average particle diameter ranges from 50 nm to 1 μm.

4. Particle according to claim 1, wherein the shell is also coated at least partly by a lamellar phase.

5. Particle according to claim 1, wherein compounds X and Y are capable of reacting by hydrosilylation in the presence of a catalyst.

6. Particle according to claim 5, wherein compound X is selected from silicone compounds comprising at least two unsaturated aliphatic groups.

7. Particle according to claim 5, wherein compound X is a polyorganosiloxane comprising a silicone main chain whose unsaturated aliphatic groups are pendent from the main chain (side group) or located at the ends of the main chain of the compound (end group).

8. Particle according to claim 7, wherein compound X bears at least one polar group.

9. Particle according to claim 5, wherein compound X is selected from the polyorganosiloxanes comprising at least two unsaturated aliphatic groups each attached to a silicon atom.

10. Particle according to claim 5, wherein compound X is selected from the polyorganosiloxanes containing siloxane units of formula:
in which:
$${R}_{m} {R}^{′} SiO ( \frac{3 - m}{2} ) \qquad ( I )$$
R represents a linear or cyclic, monovalent hydrocarbon group, having from 1 to 30 carbon atoms,
m is equal to 1 or 2 and
R′ represents:
an unsaturated aliphatic hydrocarbon group having from 2 to 10 carbon atoms or
an unsaturated cyclic hydrocarbon group having from 5 to 8 carbon atoms.

11. Particle according to claim 10, wherein the polyorganosiloxane of formula (I) is such that R′ represents a vinyl group or a group —R″—CH═CHR′″ in which R″ is a divalent aliphatic hydrocarbon chain, having from 1 to 8 carbon atoms, bound to the silicon atom and R′″ is a hydrogen atom or an alkyl radical having from 1 to 4 carbon atoms.

12. Particle according to claim 10, wherein R represents an alkyl radical having from 1 to 10 carbon atoms or alternatively a phenyl group and R′ is a vinyl group.

13. Particle according to claim 7, wherein the polyorganosiloxanes additionally comprise units of formula:
$${R}_{n} SiO ( \frac{4 - n}{2} ) \qquad ( II )$$
in which R represents a linear or cyclic, monovalent hydrocarbon group, having from 1 to 30 carbon atoms, and n is equal to 1, 2 or 3.

14. Particle according to claim 5, wherein compound X is selected from organic oligomers or polymers, hybrid organic/silicone oligomers or polymers, said oligomers or polymers bearing at least 2 unsaturated reactive aliphatic groups.

15. Particle according to claim 5, wherein compound Y comprises at least two free Si—H groups.

16. Particle according to claim 5, wherein compound Y is selected from the polyorganosiloxanes comprising at least one alkylhydrogenosiloxane unit with the following formula:
$${R}_{p} HSiO ( \frac{3 - p}{2} ) \qquad ( III )$$
in which:
R represents a linear or cyclic, monovalent hydrocarbon group, having from 1 to 30 carbon atoms or a phenyl group, and p is equal to 1 or 2.

17. Particle according to claim 16, wherein compound Y is such that the radicals R represent a C₁-C₁₀ alkyl group.

18. Particle according to claim 15, wherein Y is a polyorganosiloxane comprising at least two alkylhydrogenosiloxane units of formula —(H₃C)(H)Si—O—.

19. Particle according to claim 5, wherein the hydrosilylation is carried out in the presence of a catalyst based on platinum or tin.

20. Particle according to claim 19, wherein the catalyst represents from 0.0001% to 20% by weight relative to its total weight.

21. Particle according to claim 5, wherein compound X is a polydimethylsiloxane with vinylic end groups and compound Y is a polymethylhydrogenosiloxane.

22. Particle according to claim 1, wherein compound X bears at least one polar group that is able to form a hydrogen bond with keratin materials.

23. Particle according to claim 1, wherein compound X has a weight-average molecular weight (Mw) in the range from 150 to 1 000 000.

24. Particle according to claim 1, wherein compound Y has a weight-average molecular weight (Mw) in the range from 200 to 1 000 000.

25. Particle according to claim 1, wherein compounds X and Y are present in the particles in a molar ratio X/Y in the range from 0.05 to 20.

26. Particle according to claim 1, wherein the core comprises at least one oil.

27. Particle according to claim 1, wherein the core contains at least one lipophilic active agent other than an oil.

28. Particle according to claim 27, wherein said active agent is chosen from D-α-tocopherol, DL-α-tocopherol, D-α-tocopheryl acetate, DL-α-tocopheryl acetate, ascorbyl palmitate, vitamin F glycerides, D vitamins, vitamin D₂, vitamin D₃, retinol, retinyl esters, retinyl palmitate, retinyl propionate, carotenes, including β-carotene, D-panthenol, farnesol, farnesyl acetate, salicylic acid and derivatives thereof, for instance 5-n-octanoylsalicylic acid, alkyl esters of α-hydroxy acids such as citric acid, lactic acid, glycolic acid, asiatic acid or madecassic acid, asiaticoside, the total extract of Centella asiatica, β-glycyrrhetinic acid; α-bisabolol, ceramides, for instance 2-oleoylamino-1,3-octadecane, phytanetriol, phospholipids of marine origin rich in polyunsaturated essential fatty acids, ethoxyquine, extract of rosemary, extract of lemon balm, quercetin, extract of dried microalgae, essential oil of bergamot, octyl methoxycinnamate, butylmethoxydibenzoylmethane, octyl triazone, 3,5-di-tert-butyl-4-hydroxy-3-benzylidene-camphor, antibiotics, antifungal agents, anaesthetics, analgesics, antiseptics, antiviral agents, pesticides, herbicides, anti-inflammatory agents, antibacterial agents and antimitotic agents, and mixtures thereof.

29. Aqueous dispersion of particles according to claim 1.

30. Process for preparing particles of core/shell type, the core of which is lipophilic and the shell of which is formed at least partly from a silicone polymer film, comprising at least steps of:
i. dissolving, in at least one volatile organic solvent, at least one lipophilic compound, a compound X and a compound Y, with at least one of the compounds X and Y being an organopolysiloxane and compounds X and Y being under conditions not suitable for their interaction, and
ii. placing the organic phase from step 1 in contact with an aqueous phase containing, where appropriate, at least one surfactant, under conditions suitable for the formation of lipophilic droplets and for the reaction of compounds X and Y according to a reaction of hydrosilylation type in the presence of a catalyst, or of condensation or crosslinking type in the presence of a peroxide, to form a silicone polymer film at the surface of said droplets.

31. Process according to claim 30, wherein said particles formed are isolated via evaporation of the organic solvent(s).

32. Process according to claim 30, wherein the organic solvent is sparingly water-miscible and step 2 corresponds to an emulsification of the organic phase from step 1 in the aqueous phase in the form of droplets, at the surface of which compounds X and Y interact to form the polymer film constituting the shell of said particles.

33. Process according to claim 30, wherein the organic solvent is water-miscible and step 2 corresponds to solvent tipping, during which the diffusion of the aqueous phase into the organic phase causes the formation of droplets, at the surface of which compounds X and Y interact to produce the polymer film forming the shell of said particles.

34. Process according to claim 30, at least one of compounds X and Y being a polyorganosiloxane.

35. Process according to claim 30, wherein the organic phase of step 1 also comprises a catalyst.

36. Process according to claim 30, wherein the organic phase of step 1 also comprises a compound capable of forming a lamellar phase at the surface of said particles.

37. Cosmetic and/or therapeutic composition containing, in a physiologically acceptable medium, at least one particle according to claim 1.

38. Composition according to claim 37, wherein said particle contains at least one lipophilic agent chosen from D-α-tocopherol, DL-α-tocopherol, D-α-tocopheryl acetate, DL-α-tocopheryl acetate, ascorbyl palmitate, vitamin F glycerides, D vitamins, vitamin D₂, vitamin D₃, retinol, retinyl esters, retinyl palmitate, retinyl propionate, carotenes, including β-carotene, D-panthenol, framesol, farnesyl acetate, salicylic acid and derivatives thereof, for instance 5-n-octanoylsalicylic acid, alkyl esters of α-hydroxy acids such as citric acid, lactic acid, glycolic acid, asiatic acid or madecassic acid, asiaticoside, the total extract of Centella asiatica, β-glycyrrhetinic acid, α-bisabolol, ceramides, for instance 2-oleoylamino-1,3-octadecane, phytanetriol, phospholipids of marine origin rich in polyunsaturated essential fatty acids, ethoxyquine, extract of rosemary, extract of lemon balm, quercetin, extract of dried microalgae, essential oil of bergamot, octyl methoxycinnamate, butylmethoxydibenzoylmethane, octyl triazone and 3,5-di-tert-butyl-4-hydroxy-3-benzylidenecamphor.

39. Composition according to claim 37, wherein it is in the form of a composition for caring for and/or making up keratin material(s).

40. Cosmetic method for treating keratin material(s), comprising at least the application to a keratin material of particles according to claim 1.

41. Cosmetic method for treating keratin material(s), comprising at least the application to a keratin material of a composition according to claim 37.
